(19) United States
(12) Patent Application Publication
(10) Pub. No.: US 2026/0137302 A1
(43) Pub. Date: May 21, 2026

(54) DIGITAL ANTHROPOMETER AND METHODS OF USE

(71) Applicant: Solutions Through Innovative Technologies, Inc., Beavercreek, OH (US)

(72) Inventor: Frank Maranje, Dayton, OH (US)

(21) Appl. No.: 18/955,661

(22) Filed: Nov. 21, 2024

(51) Int. Cl. A61B 5/107; G01D 5/347

(52) U.S. Cl. CPC A61B 5/1072; G01D 5/34738; G01D 5/34792; A61B 5/1075

(57) ABSTRACT

The present disclosure pertains to digital anthropometer devices and methods of use. A portable measurement device comprises a rotary encoder housed within an enclosure. The device includes a wheel rotatably coupled to the rotary encoder, enabling displacement measurement as the wheel rolls along a surface. The measured displacement is displayed on a digital screen integrated into the enclosure. The device further includes at least one control button mounted on the enclosure, allowing the user to operate the rotary encoder and manage the measurement process.

### CROSS REFERENCE TO RELATED APPLICATIONS

Not Applicable.

### FIELD

This disclosure pertains to the field of anthropometry, and specifically, but by way of limitation, to devices and methods for measuring human body dimensions with enhanced accuracy using integrated digital components and wireless communication capabilities.

### SUMMARY

According to some embodiments, the present disclosure is directed to a digital anthropometer. The digital anthropometer also includes a rotary encoder; an enclosure housing the rotary encoder, a wheel associated with the enclosure and rotatably coupled to the rotary for measuring displacement as the wheel rolls along a surface, a digital display configured to show the displacement as a measurement value, and at least one control button on the enclosure for operating the rotary encoder. Other embodiments of this aspect include corresponding computer systems, apparatus, and computer programs recorded on one or more computer storage devices, each configured to perform the actions of the methods.

Implementations may include one or more of the following features. The digital anthropometer may include a rail engagement interface having: rollers rotatably supported on the enclosure, where the rollers engage with a measurement rail and support the digital anthropometer as the digital anthropometer travels along the measurement rail; and the wheel is configured to roll along the measurement rail, the measurement rail being placed in proximity to a body or body part of a user. The digital anthropometer may include a second digital display positioned on an opposite side of the enclosure to provide measurement visibility from multiple angles. The control button includes: a unit toggle button to switch between different measurement units of the measurement value, a hold button to freeze the measurement value displayed on the digital display, and a zero button to reset the measurement value to zero. The digital anthropometer may include a pressure sensor associated with the rotary encoder, the pressure sensor sensing an amount of force applied to the wheel during measurement. The rotary encoder is configured to measure distance with a resolution of 0.1 mm. The digital anthropometer may include a detachable component for switching between anthropometry and skinfold measurement modes. The digital anthropometer may include a mercury switch or equivalent sensor to indicate the horizontal plane orientation of the device. Implementations of the described techniques may include hardware, a method or process, or computer software on a computer-accessible medium.

One general aspect includes a method of using the digital anthropometer. The method also includes positioning the wheel on a surface to be measured, rolling the wheel along the surface to measure displacement, converting the displacement into distance using the rotary encoder, displaying the measured distance on the digital display, and transmitting the measurement data wirelessly to an external device via the Bluetooth module. Other embodiments of this aspect include corresponding computer systems, apparatus, and computer programs recorded on one or more computer storage devices, each configured to perform the actions of the methods.

Other embodiments of this aspect include corresponding computer systems, apparatus, and computer programs recorded on one or more computer storage devices, each configured to perform the actions of the methods.

Implementations may include one or more of the following features. The method may include switching between different measurement units by toggling the unit button. The method may include freezing the displayed measurement value by pressing the hold button. The method may include indicating the amount of force applied during measurement using the pressure sensor. The method may include providing measurement visibility from multiple angles using the second digital display. The method may include switching between anthropometry and skinfold measurement modes by attaching or detaching the respective component. The method may include indicating the horizontal plane orientation of the device using the mercury switch or equivalent sensor. Implementations of the described techniques may include hardware, a method or process, or computer software on a computer-accessible medium.

One general aspect includes a digital anthropometer. The digital anthropometer also includes a rotary encoder; an enclosure housing the rotary encoder, a wheel associated with the enclosure and rotatably coupled to the rotary encoder for measuring displacement as the wheel rolls along a body part of a user to measure a length of the body part, a pressure sensor associated with the rotary encoder, a microcontroller configured to: receive output from the rotary encoder indicative of the wheel being used against the body part, receive a pressure signal from the pressure sensor while the wheel being used against the body part of the user, and determine a measurement value corresponding to the length of the body part based on rotations of the wheel determined from the rotary encoder. Other embodiments of this aspect include corresponding computer systems, apparatus, and computer programs recorded on one or more computer storage devices, each configured to perform the actions of the methods.

Implementations may include one or more of the following features. The digital anthropometer may include a support arm extending from the enclosure. The pressure sensor provides real-time feedback to the microcontroller, allowing for dynamic adjustment of the displayed measurement value based on the detected pressure to ensure accurate measurements. The microcontroller is further configured to alert via the digital display or an auditory signal when an applied pressure exceeds a predefined threshold, indicating that excessive force is being applied so as to avoid distorting the measurement value. Implementations of the described techniques may include hardware, a method or process, or computer software on a computer-accessible medium.

### BRIEF DESCRIPTION OF THE DRAWINGS

FIG. 1 is a perspective view of an example device of the present disclosure in use by a first user, measuring a height of a second user.

FIG. 2 is a perspective view of the device shown in FIG. 1.

FIG. 3 is another perspective view of the device shown in FIG. 1

FIG. 4 is a perspective view of the device with parts being shown in phantom.

FIG. 5 is a cross-section view of the device in association with a measurement rail.

FIG. 6 is an example electrical circuit diagram for use in a device of the present disclosure.

FIG. 7 is a flowchart of an example method of the present disclosure.

FIG. 8 is a perspective view of another example anthropometric device in the form of a caliper.

FIG. 9 is a schematic diagram of an example computer system that can be used in accordance with the present disclosure

### DETAILED DESCRIPTION OF EXAMPLE EMBODIMENTS

### Overview

The digital anthropometer described here is an innovative tool designed to address the longstanding challenges in anthropometry, which include the need for accurate and consistent measurement of human body dimensions. Traditional tools often struggle with manual inaccuracies, lack of digital integration, and limited functionality, all of which can lead to inefficiencies and errors in data collection. The digital anthropometer disclosed herein provides a technical solution by integrating advanced digital technologies and features that greatly enhance the precision, usability, and data handling capabilities of anthropometric measurements.

One of the primary issues with traditional anthropometric tools is the reliance on manual operations, which are susceptible to human error and inconsistencies. Additionally, these tools often do not integrate well with digital systems, limiting their ability to store, analyze, and share data efficiently. The digital anthropometer addresses these issues by incorporating a rotary encoder connected to a wheel, which accurately measures displacement as the wheel rolls along a surface. This configuration ensures that the measurements are consistent and precise, significantly reducing the likelihood of errors associated with manual measurements.

To further enhance measurement accuracy and consistency, the digital anthropometer features a rail engagement interface designed to work seamlessly with a measurement rail. This interface includes components such as rollers that engage with the rail, ensuring that the device maintains a stable and aligned path as it moves along the surface. The measurement rail itself is a rigid, straight structure that guides the device, minimizing deviations and maintaining constant contact between the wheel of the rotary encoder and the rail. This combination of the rail engagement interface and measurement rail significantly improves the precision of measurements, particularly over long distances or on irregular surfaces, making the anthropometer highly reliable and effective in various professional applications.

The digital anthropometer is also equipped with an integrated digital display that provides real-time feedback on measurements, allowing users to instantly verify and adjust as needed. This feature is particularly valuable when measuring individuals of varying heights, as the device includes a “hold” function that allows the user to freeze the measurement display while adjusting the position of the device for easier viewing. The anthropometer's user-friendly design includes control buttons strategically placed on the enclosure, making it straightforward to operate the device, even in challenging measurement scenarios.

In addition to improving measurement accuracy and ease of use, the digital anthropometer offers advanced data integration and connectivity options. For instance, it features Bluetooth connectivity, allowing measurement data to be easily transmitted to computers or other devices for further analysis and record-keeping. This capability is especially beneficial for fields that require extensive data processing, such as ergonomic studies, healthcare, and custom clothing design. Furthermore, the device can be integrated into larger systems, such as medical records or ergonomic databases, enhancing its utility across various industries.

The design of the digital anthropometer also emphasizes flexibility and modularity. The device is built in such a way that components can be easily swapped or upgraded, allowing it to adapt to different measurement scenarios. For example, different wheels or sensors can be attached to the rotary encoder to measure different body parts or accommodate different surfaces. This modularity extends to the device's software as well, which can be updated to incorporate new features or improved algorithms for measurement accuracy.

In sum, the digital anthropometer represents a significant advancement in the field of anthropometry. It offers a more accurate, user-friendly, and technologically integrated solution for measuring human body dimensions, addressing the limitations of traditional tools and providing new opportunities for data integration and analysis.

### EXAMPLE EMBODIMENTS

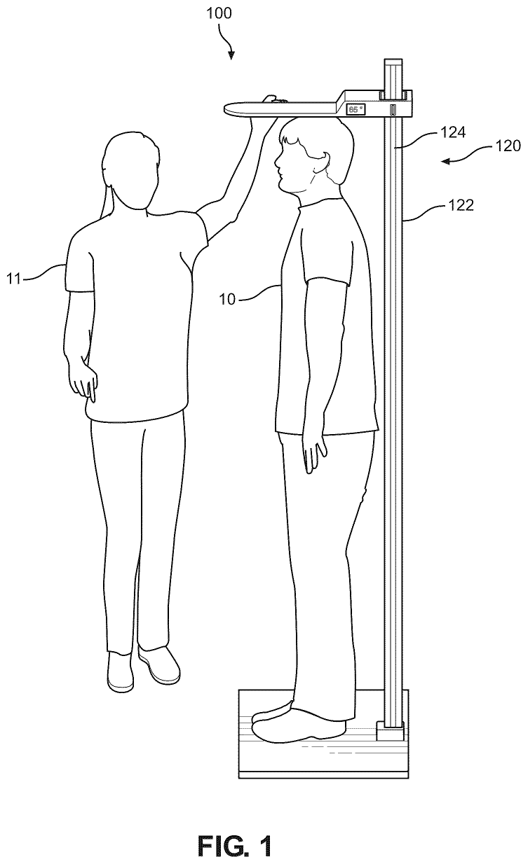

FIG. 1 provides a perspective view of a digital anthropometer 100 (device 100) in accordance with the present disclosure. The device 100 is broadly described as a digital anthropometer and is designed to measure the physical dimensions of a person 10 by a user 11.

Though the device 100 is shown in combination with a measurement rail 120 in this illustration, the device 100 can be used without the measurement rail 120. In operation, the user 11 can employ the device 100 to capture various anthropometric measurements, such as height, limb length, or other body dimensions, with a high degree of precision. To begin the measurement process, the user 11 positions the device 100 at a starting point along the dimension of the body of the person 10 to be measured. Depending on the specific dimension being measured, the user 11 may align the device 100 vertically, horizontally, or at an angle relative to the person's body. The design of the device 100 allows it to be easily maneuvered by the user 11 to accommodate different measurement requirements, ensuring flexibility and ease of use in a variety of settings.

Once the device 100 is properly positioned, the user 11 operates the device to capture the measurement by carefully guiding it along the path of the dimension to be measured. Throughout this process, the user 11 can rely on the device's digital interface to monitor the measurement in real-time, allowing for adjustments to be made as necessary to ensure accuracy. The ergonomic design of the device 100 facilitates ease of handling, providing the user 11 with precise control during the measurement process. This is particularly useful in settings where multiple measurements must be taken quickly and efficiently, such as in clinical environments, research studies, or ergonomic assessments.

The digital anthropometer 100 enhances traditional measurement methods by reducing the potential for human error and providing the user 11 with immediate feedback on the measurement. Additionally, the device 100 can be employed in various professional fields, such as healthcare, ergonomics, sports science, and apparel design, where accurate anthropometric data is crucial. By allowing the user 11 to take precise, repeatable measurements, the device 100 streamlines the process of body dimension assessment, offering an efficient and user-friendly solution to the challenges commonly encountered with manual measurement tools.

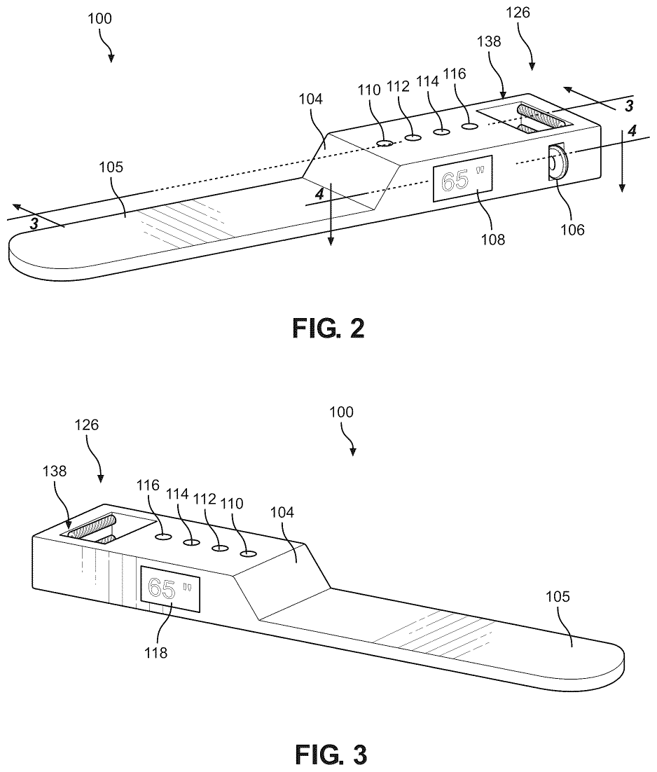

Referring now to FIGS. 2-4 collectively, the device 100 can include a rotary encoder 102, an enclosure or housing 104, a wheel 106, and a first display 108. The enclosure 104 of the digital anthropometer 100 is a protective housing that encases and secures all the internal components, including a rotary encoder, microcontroller, displays, and control buttons. The enclosure 104 is designed not only to protect these sensitive electronics from physical damage, dust, and moisture but also to provide a comfortable and ergonomic interface for the user. It typically features smooth contours and a design that fits comfortably in the hand, making it easy to maneuver the device along a surface or body part during measurement.

Enclosure 104 can be made from a variety of materials, each chosen based on factors like durability, weight, and cost. Common materials include high-impact plastics such as ABS (Acrylonitrile Butadiene Styrene), which is known for its toughness, impact resistance, and ease of manufacturing. ABS plastic is lightweight yet strong, making it ideal for portable devices that need to withstand everyday use. Another option is polycarbonate, a material that offers even greater strength and transparency, allowing for more durable and aesthetically pleasing designs. In some cases, the enclosure 104 might be made from aluminum or other metals, which provide superior protection against impact and wear. For specialized applications where sterility and resistance to harsh chemicals are important, the enclosure 104 might be constructed from medical-grade materials, such as stainless steel or certain types of polymers. These materials ensure that the device can be safely used in environments like hospitals or laboratories without compromising its longevity or performance.

In some instances, the device 100 includes a support arm 105. The support arm 105 of the digital anthropometer 100 is an integral component designed to provide additional stability and control during the measurement process. This arm extends from the enclosure 104, offering a rigid structure that helps a user guide the device 100 as the device 100 moves along the surface being measured. The support arm 105 plays a role in maintaining the alignment of the device, ensuring that the wheel 106 of the rotary encoder 102 remains in consistent contact with the surface, thereby improving measurement accuracy and reliability.

The support arm 105 can be made from various materials, depending on the requirements for strength, weight, and flexibility. Common materials include lightweight metals like aluminum, which offers a strong yet manageable support structure without adding excessive weight to the device. Aluminum is also resistant to corrosion, making it suitable for prolonged use in different environments. In some cases, the support arm 105 might be constructed from high-strength plastics or composite materials, such as carbon fiber, which provide a superior strength-to-weight ratio and enhance the portability of the device. These materials ensure that the support arm 105 can withstand the stresses of repeated use while maintaining the precise alignment necessary for accurate measurements.

As noted above, the device 100 is a versatile and portable device designed for precise measurement of human body dimensions in a wide range of settings. By itself, the device 100 can be employed in various fields such as healthcare, ergonomics, sports science, and clothing design. Its compact and user-friendly design allows it to be easily handled and operated by professionals and laypersons alike. The device's core functionality revolves around the rotary encoder 102, which, when moved along a surface, measures displacement of the device 100 with high accuracy. The results are displayed in real-time on an integrated digital screen (first display 108), enabling immediate verification and recording of measurements.

In a clinical setting, for example, healthcare professionals can use the device 100 to accurately measure a patient's limb lengths, body circumference, or other anthropometric data critical for assessing growth patterns, diagnosing conditions, or planning treatments. In ergonomics, the device 100 can be used to measure body dimensions to tailor workstations or tools to fit individual users, thereby enhancing comfort and reducing the risk of injury. Similarly, in the clothing industry, designers can use the device 100 to obtain precise body measurements for custom tailoring or to ensure that off-the-rack clothing fits a wide range of body types.

The device 100 allows for the straightforward and precise measurement of a person's body dimensions by rolling the wheel 106 along the part of the body or body part to be measured. Whether measuring limb lengths, torso circumference, or the distance between specific body points, the wheel 106 can be translated along the surface of the skin or clothing, capturing accurate displacement data in real time. The ergonomic design of the digital anthropometer 100 ensures that it moves smoothly over the body's contours, while the integrated digital display provides immediate feedback, allowing users to monitor the measurement process and make any necessary adjustments on the spot. This simple yet effective method of rolling the wheel 106 along the desired body part eliminates the need for complex setups or manual calculations, making the device 100 an ideal tool for quick and reliable anthropometric measurements in various settings such as clinical assessments, ergonomic studies, or custom clothing fittings.

In more detail, the rotary encoder 102 is integrated into the enclosure 104 and can be coupled to the wheel 106 via a shaft 107. A rotary encoder is an electromechanical device designed to convert the angular position or motion of a rotating shaft into a digital signal, which can then be used for various measurement or control applications. It operates by detecting the rotation of a shaft and converting that rotational movement into a series of electronic signals, which correspond to specific angular positions or increments. This functionality makes rotary encoders essential in systems that require precise control and measurement of rotational movement, such as in motors, robotics, or even in digital anthropometers like the one described earlier.

The rotary encoder typically consists of a shaft, a disc, a sensor, and associated electronic circuitry (such as a microcontroller discussed in greater detail herein). The shaft is the part of the device that rotates and is mechanically linked to the object or surface being measured. Attached to this shaft is a disc, which is marked with patterns such as slots, holes, or reflective segments. As the disc rotates with the shaft, the sensor detects the changes in these patterns. For instance, in an optical rotary encoder, the sensor might detect light passing through the slots on the disc. The microprocessor then processes these signals, converting them into a digital output that represents the rotational position, speed, or direction of the shaft.

There are two primary types of rotary encoders: incremental and absolute. Incremental rotary encoders generate a series of pulses as the shaft rotates, with each pulse representing a specific increment of rotation. The number of pulses counted over time corresponds to the angle of rotation, and by tracking these pulses, the system can determine the position and speed of the rotation. In contrast, absolute rotary encoders provide a unique digital code for each specific angular position of the shaft, allowing for more precise and direct measurement of position without the need for counting pulses. Both types of encoders are widely used in various applications, offering high accuracy and reliability in motion control systems.

In the device 100, the rotary encoder 102 is rotatably connected to the wheel 106, which functions as the primary measuring element of the device 100. As the wheel 106 rolls along a surface—whether it be a body part or the measurement rail—the wheel 106 rotates the shaft of the rotary encoder 102. This rotation is tracked by the rotary encoder 102, which then converts the wheel's 106 rotational movement into digital signals. These signals are processed by a microprocessor to represent the displacement or distance the wheel 106 has traveled. The processed data is then displayed on the first display 108, allowing for precise and accurate measurement readings. This integration of the rotary encoder 102 with the wheel 106 ensures that every movement of the digital anthropometer 100 is captured with high accuracy.

In a broader embodiment of the digital anthropometer 100, a control button 110 is designed to operate the rotary encoder 102, giving the user straightforward control over the measurement process. When the control button 110 is pressed, a microprocessor activates the rotary encoder 102, allowing it to begin tracking the rotation of the wheel 106 as the wheel 106 moves along a surface. This configuration ensures that the device 100 only measures when the user intends, providing precision and control over the data collection. The simplicity of this control mechanism is particularly useful in scenarios where precise timing of measurements is desired, such as when the device 100 needs to be positioned correctly before measurement begins.

In other embodiments, the device 100 includes control buttons that are equipped with functionalities to enhance the user experience and measurement accuracy. Specifically, the device 100 also includes a unit toggle button 112, a hold button 114, and a zero button 116, each designed to perform specific tasks.

The unit toggle button 112 allows the user to switch between different units of measurement, such as millimeters, centimeters, or inches. This feature is particularly useful in diverse applications where measurements need to be recorded in various units depending on specific requirements or regional standards. By pressing the unit toggle button 112, users can easily adjust the display to their preferred unit, ensuring that the measurement data is immediately relevant and applicable to the task at hand.

The hold button 114 provides the functionality to freeze the current measurement value displayed on the digital display 108. This feature is useful when the user needs to record or review a measurement without the risk of altering the displayed value through unintended movement. By pressing the hold button 114, the user can lock the measurement on the display, allowing for careful documentation or analysis before continuing with further measurements.

The zero button 116 is designed to reset the measurement value to zero, providing a clean slate for new measurements. This button is important for maintaining accuracy and consistency, as it ensures that each new measurement starts from a baseline, free from any residual values from previous operations. The zero button 116 allows the user to recalibrate the device 100 quickly and easily, ensuring that all measurements are accurate and reliable.

The digital anthropometer 100 is equipped with two digital displays, with the first display 108 positioned on one side of the enclosure 104 and the second display 118 (see FIG. 3) located on the opposite side of the enclosure 104 relative to the first display 108. This dual-display configuration significantly enhances the usability and versatility of the device, allowing for improved visibility of measurement data in various scenarios.

The first display 108 serves as the main interface for a user, providing clear and immediate feedback on the measurements as they are being taken. However, in situations where the orientation of the device 100 makes it difficult for the user to view the first display 108—such as when the device is positioned at an awkward angle or when the user needs to view the data from a different perspective—the second display 118 provides an additional viewpoint. The second display 118 ensures that the measurement data remains easily accessible and visible, regardless of how the device 100 is oriented or positioned during use.

This feature is particularly beneficial in environments where multiple measurements need to be taken quickly and efficiently, or where the user must frequently adjust the device's position. For example, in ergonomic assessments or clinical settings, the second display 118 allows the user to maintain a clear view of the measurement data without needing to reposition the device 100 or themselves, thereby reducing measurement time and minimizing the potential for errors.

Furthermore, the second display 118 provides added convenience in collaborative settings, where multiple users may need to view the measurement data simultaneously from different angles. By offering visibility from both sides of the enclosure 104, the dual-display setup ensures that all relevant parties can monitor the measurement process in real time, facilitating better communication and decision-making.

In summary, the inclusion of the second display 118 on the digital anthropometer 100 enhances the device's functionality by ensuring that measurement data is always visible and accessible, regardless of the device's orientation. This dual-display feature not only improves user convenience but also contributes to more efficient and accurate measurements in various professional settings.

### Digital Anthropometer and Measurement Rail

Referring briefly back to FIG. 1, when used in conjunction with the measurement rail 120, the device 100 functionality is enhanced, allowing for even more precise and consistent measurements over longer distances or across larger surfaces. The measurement rail typically serves as a stable guide along which the anthropometer can be moved. This arrangement is beneficial for tasks that require measurements over extended body lengths, such as the height of a person, the length of a limb, or the span of a shoulder width.

The measurement rail 120 ensures that the device 100 remains aligned correctly, minimizing the potential for deviation or error that might occur if the device 100 were used freehand. Similarly, in the design of ergonomic workspaces or vehicles, the combination of the digital anthropometer and measurement rail 120 allows for the precise mapping of body dimensions to optimize seat configurations, control placements, and other critical ergonomic factors.

Moreover, the measurement rail 120 can be modular, allowing it to be adjusted or extended to accommodate different measurement tasks. The measurement rail 120 could also be equipped with additional sensors or markers that interact with the digital anthropometer to automatically record measurements at predefined intervals or locations. This automation can streamline data collection in large-scale studies or industrial applications, where consistency and speed are paramount.

The measurement rail 120 includes several components that work together to ensure accurate and consistent measurements. A rail body 122 is a rigid, elongated structure made from durable materials like aluminum, steel, or reinforced plastic. This rail body 122 provides a stable, straight surface along which the device 100 can move.

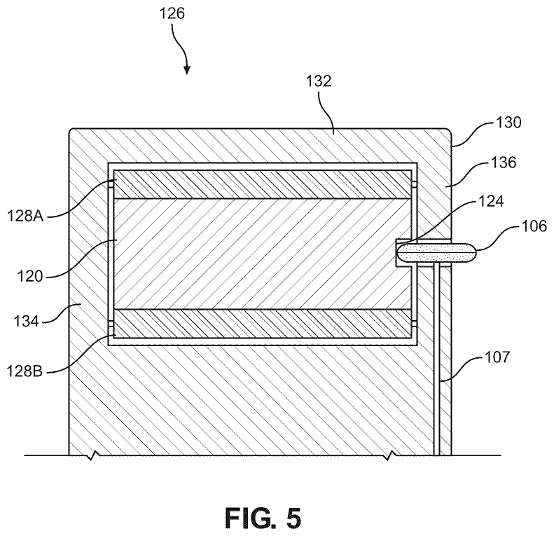

As best illustrated in FIGS. 1 and 5, integrated into the rail body 122 is a guide track 124, a groove or set of raised edges designed to engage with the device 100. The guide track plays a role in maintaining the alignment of the device 100 as it travels along the rail, ensuring that the wheel 106 of the rotary encoder 102 remains in consistent contact with the rail's surface. This contact captures precise displacement measurements, as it minimizes any potential deviations or errors that could arise from misalignment. Together, these components of the measurement rail provide the necessary structure and guidance to facilitate accurate, reliable anthropometric measurements.

The digital anthropometer 100 incorporates a system of rollers designed to work seamlessly with the measurement rail 120, ensuring that the rotary encoder's wheel remains in continuous, accurate contact with the rail throughout the measurement process. This design is used for maintaining the precision of measurements, particularly over long distances or uneven surfaces where the risk of wheel slippage or misalignment could compromise the accuracy of the readings.

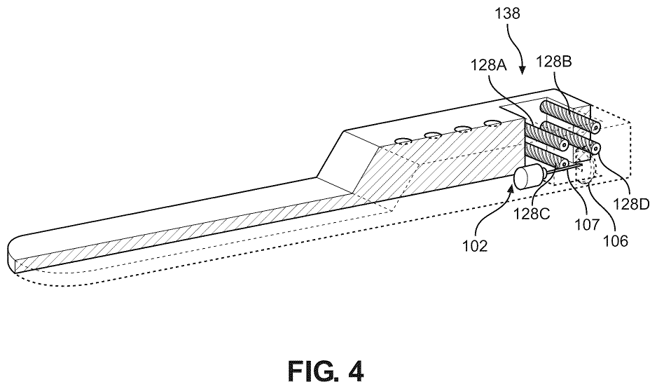

As best illustrated in FIGS. 4 and 5, the device 100 can include a rail engagement interface 126 that allows the device 100 to intermesh with the measurement rail 120. The rail engagement interface 126 can include a plurality of rollers, and in some instances, four rollers 128A-128D that are each rotatably supported by the enclosure 104.

As the digital anthropometer 100 is moved along the measurement rail 120, the rollers 128A-128D, typically made from high-friction materials such as rubber, are strategically positioned to press against the sides of the rail, and specifically the guide track 124. This configuration effectively allows the device 100 to grip the measurement rail 120, keeping the digital anthropometer 100 aligned and the wheel 106 securely in place. The constant pressure exerted by the rollers 128A-128D ensures that the wheel 106 of the rotary encoder 102 remains in contact with the measurement rail 120, preventing any interruptions that might lead to inaccurate displacement readings. This continuous contact ensures precise measurements, as any deviation could result in errors.

The integration of the rollers 128A-128D with the measurement rail 120 offers several significant benefits. Firstly, it guarantees consistent contact between the rotary encoder's wheel and the rail, eliminating the possibility of slippage or lift-off, which are common issues that can lead to measurement inaccuracies. Secondly, the rollers help maintain the alignment and stability of the digital anthropometer 100 as it travels along the measurement rail 120, ensuring that measurements are taken along a straight, predefined path.

Moreover, the rollers 128A-128D enable smooth operation of the digital anthropometer 100 along the measurement rail 120, reducing friction and wear on both the device and the measurement rail 120. This smooth movement helps achieve steady measurements without abrupt stops or jerks that could otherwise affect accuracy. The roller system is also adaptable to different types of rails, allowing digital anthropometer 100 to be used in various measurement scenarios, whether the measurement rail 120 is mounted horizontally, vertically, or at an angle.

In some instances, the rail engagement interface 126 includes a frame 130 that extends from one end of the enclosure 104. For example, the frame 130 can include an end 132 and opposing sidewalls 134 and 136. When assembled, the frame 130 defines a pass-through 138 (see FIGS. 2 and 3).

The rollers 128A-128 are supported on a terminal end of the enclosure 104 and extend from the enclosure and engage rotatably with the end 132 of the frame 130. The device 100 is engaged with the measurement rail 120 by passing the device 100 over an end of the measurement rail 120. The measurement rail 120 engages with the rollers 128A-128 and the wheel 106 contacts the measurement rail 120. The rollers 128A-128 maintain a desired orientation between the rollers 128A-128 and the wheel 106, relative to the measurement rail 120.

In some arrangements, the wheel 106 is configured to travel within the guide track 124 of the measurement rail 120. The guide track 124 could be a groove or notch fabricated into the rail body 122. In other instances, the guide track 124 could be defined by shoulders that extend along the guide track 124.

As can be seen, the wheel 106 also extends partly beyond the sidewall 136 of the frame 130 so that the device 100 can be used without the measurement rail 120. That is, a portion of the wheel 106 extends past an outer perimeter of the frame 130 and a portion of the wheel 106 is inside the frame 130. This orientation of the wheel 106 allows the device to be used with or without the measurement rail 120.

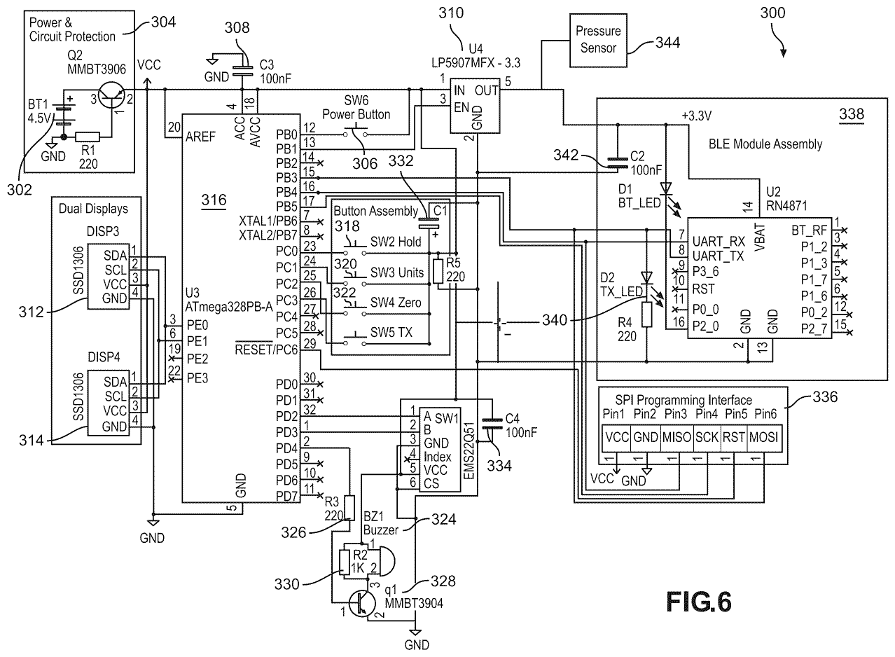

Referring now to FIG. 6, which illustrates an example circuit diagram 300 of an example digital anthropometer. In some instances, a power source 302, represented by the battery BT1, supplies the necessary electrical energy to power the entire circuit of the digital anthropometer. This component typically provides a stable voltage, crucial for the consistent operation of the device. In this circuit, the battery is likely a 4.5V source, ensuring sufficient power to drive the microcontroller, displays, sensors, and communication modules. The power source 302 is fundamental, as it initiates and sustains the operation of all other components within the system.

The power and circuit protection 304, which includes the transistor Q2 (MMBT3906) and resistor R1, is designed to protect the circuit from potential overvoltage, short circuits, or other electrical anomalies that could damage sensitive components. The transistor operates as a switch, controlling the flow of power in the circuit, while the resistor limits current to safe levels. This protection circuit ensures that the digital anthropometer can operate reliably without the risk of electrical damage, thereby extending the device's longevity and reliability.

The power button 306, labeled SW6, is the user interface element that allows the user to turn the device on and off. This button controls the connection between the battery and the rest of the circuit, enabling the user to activate or deactivate the device as needed. When pressed, it closes the circuit, allowing current to flow from the power source 302 through the rest of the system. This button conserves battery life and providing user control over when the device is operational.

Capacitor C3 308, with a value of 100 nF, is placed in the circuit to stabilize the power supply. This capacitor smooths out any fluctuations in voltage from the power source 302, ensuring a consistent and stable power supply to the rest of the circuit. This stability is important for the microcontroller and sensitive sensors, as fluctuations could lead to errors in measurement or operation. By filtering out noise and transient voltages, the capacitor helps maintain the overall reliability of the digital anthropometer.

The voltage regulator 310, identified as U4 (LP5907MFX-3.3), is responsible for converting the battery voltage to a steady 3.3V, which is the operating voltage for most components in the circuit, including the microcontroller and displays. This regulator ensures that all parts of the system receive a consistent voltage, which is crucial for accurate and reliable performance. Without this regulation, the varying battery voltage could lead to unstable operation, potentially causing measurement inaccuracies or communication failures.

The first digital display 312 (see first display 108 of FIG. 1), represented by DISP3 (SSD1306), is one of the two screens that provide real-time feedback to the user. This display shows the measurement data, such as displacement values, in a clear and accessible manner. Located on one side of the enclosure, it allows the user to monitor the data as they operate the device. The SSD1306 is a common OLED display driver, known for its sharp contrast and low power consumption, making it ideal for portable devices like the digital anthropometer.

The second digital display 314 (see second display 118), identified as DISP4 (SSD1306), is positioned on the opposite side of the enclosure from the first display. This second display provides additional flexibility by allowing the user or another observer to view the measurement data from a different angle or position. This feature is particularly useful in scenarios where the device is positioned in a way that obscures one of the displays. The inclusion of two displays ensures that measurement data is always visible, regardless of the device's orientation or the user's position.

The microcontroller 316, represented by U3 (ATmega328PB-A), serves as the brain of the digital anthropometer. It processes the signals from the rotary encoder, control buttons, and other sensors, and it controls the displays and communication modules. The ATmega328PB-A is a versatile microcontroller with sufficient processing power to handle the device's tasks, including data processing, user input management, and wireless communication. It is also programmable, allowing for firmware updates and customization of the device's functions.

The hold button 318 (see hold button 114), labeled SW2, allows the user to freeze the current measurement on the digital displays 312 and 314. This function is crucial when a precise measurement needs to be recorded or analyzed without the risk of it changing due to movement. By pressing the hold button 318, the user can lock the displayed value, making it easier to document or review the data. This feature enhances the usability of the device, especially in dynamic measurement environments where stability is key.

The units button 320 (see unit toggle button 112), identified as SW3, enables the user to switch between different units of measurement, such as millimeters, centimeters, or inches. This flexibility is important for accommodating different user preferences or measurement standards depending on the application. The units button 320 allows for quick adjustments, ensuring that the displayed data is in the most relevant format for the user's needs. This feature enhances the device's versatility, making it suitable for a wide range of applications across various fields.

The zero button 322 (see zero button 116), labeled SW4, is used to reset the measurement value displayed on the digital screens to zero. This function is used when starting a new measurement, as it ensures that the device begins from a known reference point, free from any previous data. By pressing the zero button 322, the user can recalibrate the device quickly, ensuring accurate and consistent measurements. This feature is particularly useful in situations where multiple measurements are taken in succession, as it helps maintain accuracy throughout the process.

The buzzer 324, represented by BZ1, provides auditory feedback to the user, often signaling when a button has been pressed or when a measurement has been successfully taken. This auditory signal helps confirm actions, particularly in environments where visual feedback might be insufficient, such as in noisy or low-visibility conditions. The buzzer 324 enhances the user interface by providing additional cues, making the device easier to use without requiring constant visual attention.

Resistor R3 326, with a value of 220 Ω, can limit the current flowing through certain parts of the circuit, such as the buzzer or LEDs, protecting these components from potential damage due to excessive current. By controlling the current flow, resistor R3 326 ensures that all components operate within their specified parameters, thereby extending the life of the device and maintaining consistent performance.

The transistor 328, labeled Q1 (MMBT3904), is used to amplify or switch electronic signals within the circuit. In this context, it acts as a switch to control the operation of the buzzer or other components, allowing them to be turned on or off in response to signals from the microcontroller 316. The transistor 328 enables efficient control of these components, contributing to the overall functionality and responsiveness of the device.

Resistor R2 330, with a value of 1 kΩ, is used in conjunction with the transistor 328 to further control the current and voltage levels within the circuit. This resistor ensures that the transistor operates correctly, providing the necessary biasing to switch the connected components on or off. Resistor R2 330 plays a crucial role in the stability and reliability of the switching operations within the digital anthropometer.

Capacitor C1 332, another 100 nF capacitor, can be used to filter out noise from the power supply or signal lines, ensuring clean and stable operation of the microcontroller 316 and other sensitive components. This capacitor helps to smooth out voltage fluctuations that could otherwise interfere with the accurate processing of signals, contributing to the overall reliability of the device.

Capacitor C4 334, also rated at 100 nF, serves a similar function to C1, providing additional filtering and stabilization for the circuit. By placing these capacitors strategically throughout the circuit, the design ensures that all components receive clean, stable power and signals for maintaining accurate measurements and reliable communication.

The SPI programming interface 336, represented by the pin headers, allows for direct programming of the microcontroller 316. This interface is used during the initial setup and for any subsequent firmware updates or modifications. The SPI interface provides a reliable and efficient way to upload code to the microcontroller, enabling customization of the device's functions and features. This capability is important for maintaining the device's relevance and functionality over time, as it allows for easy updates and improvements.

The Bluetooth module 338, labeled U2 (RN4871), enables wireless communication between the digital anthropometer and external devices, such as smartphones, tablets, or computers. This module allows measurement data to be transmitted in real-time, facilitating remote monitoring, data logging, and further analysis. The Bluetooth module 338 enhances the device's versatility by integrating it into a broader digital ecosystem, making it easier to use in various professional and research settings.

The status LED 340, identified as D2 (TX_LED), provides visual feedback on the operation of the Bluetooth module 338. This LED typically indicates when the module is powered on, when it is transmitting data, or when it has established a connection with an external device. The status LED 340 helps the user monitor the communication status at a glance, ensuring that the data is being transmitted correctly and that the device is functioning as intended.

In some embodiments, a capacitor C2 342, another 100 nF component, is used to stabilize the power supply specifically for the Bluetooth module 338. This capacitor filters out any noise or voltage spikes that could disrupt the wireless communication, ensuring that the Bluetooth module 338 operates smoothly and reliably. By maintaining a stable power supply, capacitor C2 342 plays a crucial role in the consistent performance of the wireless features of the digital anthropometer.

Referring now to FIGS. 1 and 6, the circuit 300 can also include a pressure sensor 344. The pressure sensor 344 is designed to enhance the accuracy and reliability of the measurements taken by the device. This pressure sensor 344 is strategically positioned near the rotary encoder 102 and wheel 106, where the pressure sensor 344 detects the amount of force applied to the wheel 106 as the wheel 106 rolls along a surface. By measuring this force, the pressure sensor 344 ensures that the wheel 106 maintains consistent contact with the surface to obtain precise displacement readings.

When the device 100 is in use, the pressure sensor 344 continuously monitors the pressure exerted on the wheel 106. This data is then sent to the microcontroller 316, which processes the information in real time. If the sensor detects that the applied pressure exceeds a certain threshold, indicating that excessive force is being applied, the microcontroller can take corrective actions. For example, the microcontroller 316 can adjust the measurement data to account for the added force or alert the user via the digital display 312 or 314, or even through the buzzer 324, that too much pressure is being applied. This feedback helps prevent inaccurate measurements that could result from the wheel being pressed too hard against the surface, which could cause slippage or distort the displacement readings.

Moreover, the pressure sensor 344 adds an extra layer of functionality to the device 100 by enabling more sophisticated measurement techniques. For instance, in scenarios where different surfaces or body parts might require varying amounts of pressure for accurate readings, the sensor can help calibrate the device accordingly. It can also be used to ensure that the measurement process is consistent across different operators, as the device can be configured to alert users when the pressure deviates from an optimal range or alternative when an appropriate level of pressure is being used.

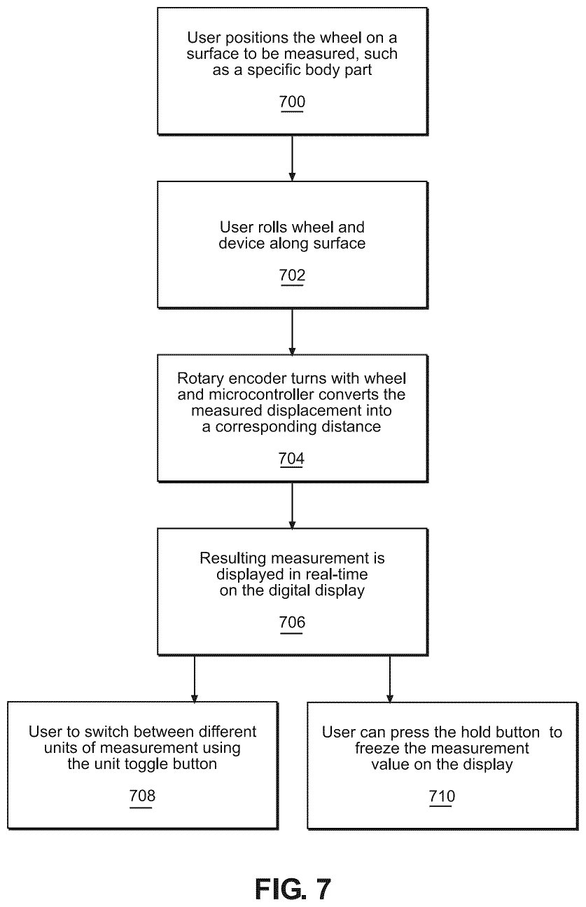

Turning now to FIG. 7, which describes a method for using a digital anthropometer of the present disclosure. The method of using the digital anthropometer 100 begins with step 400, where the user positions the wheel 106 on the surface to be measured, such as a specific body part. This initial step ensures that the device is properly aligned to capture accurate measurements from the intended area.

In step 702, once the wheel 106 is in place, the user rolls it along the surface. During this movement, the rotary encoder 102 measures the displacement, accurately capturing the distance the wheel travels over the surface.

Step 704 involves the rotary encoder turning and microcontroller converting the measured displacement into a corresponding distance. This conversion is essential as it translates the raw data from the wheel's movement into meaningful measurement values.

In step 706, the resulting measurement is displayed in real-time on the digital display 210 or 212, providing the user with immediate feedback. This allows for a quick assessment of the measurement and adjustments if necessary.

Step 708 allows the user to switch between different units of measurement using the unit toggle button 112. This feature ensures that the data is presented in the most relevant format, depending on the user's specific requirements or preferences.

In another example, in step 710, the user can press the hold button 114 to freeze the measurement value on the display. This function is particularly useful for recording or analyzing the data without the risk of the value changing due to further movement of the device.

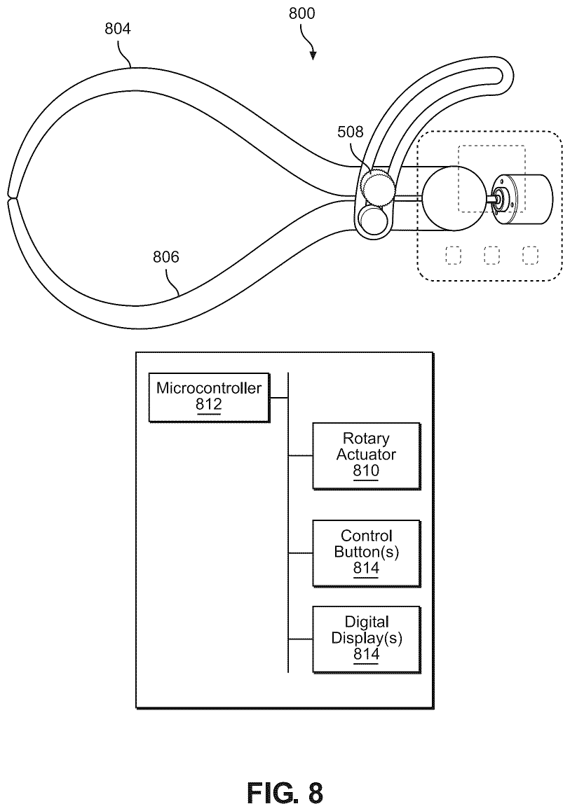

Referring now to FIG. 8, in this instance, the digital anthropometer 800 is embodied with a rotary actuator integrated into the hinge of a caliper, allowing for automated adjustment of the caliper's jaws. This configuration enables precise, consistent measurements of body dimensions with minimal manual intervention, significantly improving both accuracy and efficiency.

The caliper assembly comprises a traditional caliper mechanism adapted for anthropometric measurements. It includes two arms 804 and 806 that can be adjusted to measure the distance between two points on a body part, such as the width of a limb or the diameter of a head. These jaws move smoothly along the caliper's arms, ensuring accurate alignment and measurement.

The hinge 808 is the central pivot point of the caliper assembly, where the two arms 804 and 806 of the caliper are joined. In this embodiment, the hinge mechanism 808 is modified to incorporate a rotary actuator 810, which allows the caliper's jaws to be automatically adjusted. This actuator precisely controls the opening and closing of the jaws, eliminating the need for manual adjustments and reducing the potential for measurement errors caused by inconsistent pressure or positioning.

The rotary actuator 810 provides the necessary force to adjust the caliper's arms. It is powered and controlled by a microcontroller 812, which ensures that the actuator operates smoothly and with precision. The microcontroller 812 receives input from the control buttons 814, allowing the user to set the desired measurement parameters, such as the jaw's opening width or the force applied during measurement.

Additionally, a digital display 816 on the anthropometer provides real-time feedback on the caliper's position and the measurements being taken. This feedback is important for ensuring that the measurements are accurate and that the device is functioning correctly. The integration of the linear actuator into the hinge 808 not only enhances the functionality of the digital anthropometer 800 but also makes it a more versatile tool for a wide range of anthropometric measurements.

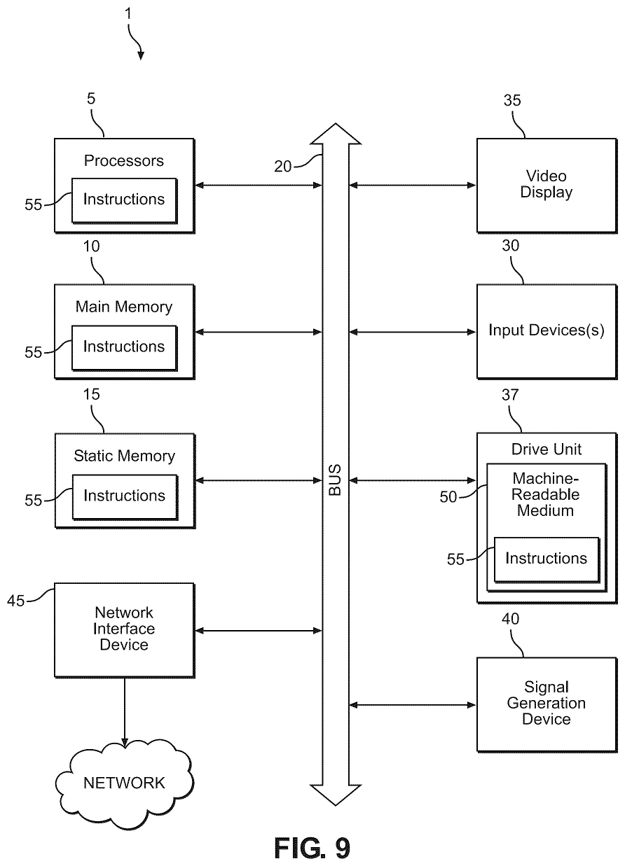

FIG. 9 is a diagrammatic representation of an example machine in the form of a computer system 1, within which a set of instructions for causing the machine to perform any one or more of the methodologies discussed herein may be executed. In various example embodiments, the machine operates as a standalone device or may be connected (e.g., networked) to other machines. In a networked deployment, the machine may operate in the capacity of a server or a client machine in a server-client network environment, or as a peer machine in a peer-to-peer (or distributed) network environment. The machine may be a personal computer (PC), a tablet PC, a set-top box (STB), a personal digital assistant (PDA), a cellular telephone, a portable music player (e.g., a portable hard drive audio device such as a Moving Picture Experts Group Audio Layer 3 (MP3) player), a web appliance, a network router, switch or bridge, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The computer system 1 includes a processor or multiple processor(s) 5 (e.g., a central processing unit (CPU), a graphics processing unit (GPU), or both), and a main memory 10 and static memory 15, which communicate with each other via a bus 20. The computer system 1 may further include a video display 35 (e.g., a liquid crystal display (LCD)). The computer system 1 may also include an alpha-numeric input device(s) 30 (e.g., a keyboard), a cursor control device (e.g., a mouse), a voice recognition or biometric verification unit (not shown), a drive unit 37 (also referred to as disk drive unit), a signal generation device 40 (e.g., a speaker), and a network interface device 45. The computer system 1 may further include a data encryption module (not shown) to encrypt data.

The drive unit 37 includes a computer or machine-readable medium 50 on which is stored one or more sets of instructions and data structures (e.g., instructions 55) embodying or utilizing any one or more of the methodologies or functions described herein. The instructions 55 may also reside, completely or at least partially, within the main memory 10 and/or within the processor(s) 5 during execution thereof by the computer system 1. The main memory 10 and the processor(s) 5 may also constitute machine-readable media.

The instructions 55 may further be transmitted or received over a network via the network interface device 45 utilizing any one of a number of well-known transfer protocols (e.g., Hyper Text Transfer Protocol (HTTP)). While the machine-readable medium 50 is shown in an example embodiment to be a single medium, the term “computer-readable medium” should be taken to include a single medium or multiple media (e.g., a centralized or distributed database and/or associated caches and servers) that store the one or more sets of instructions. The term “computer-readable medium” shall also be taken to include any medium that is capable of storing, encoding, or carrying a set of instructions for execution by the machine and that causes the machine to perform any one or more of the methodologies of the present application, or that is capable of storing, encoding, or carrying data structures utilized by or associated with such a set of instructions. The term “computer-readable medium” shall accordingly be taken to include, but not be limited to, solid-state memories, optical and magnetic media, and carrier wave signals. Such media may also include, without limitation, hard disks, floppy disks, flash memory cards, digital video disks, random access memory (RAM), read only memory (ROM), and the like. The example embodiments described herein may be implemented in an operating environment comprising software installed on a computer, in hardware, or in a combination of software and hardware.

Where appropriate, the functions described herein can be performed in one or more of hardware, software, firmware, digital components, or analog components. For example, the encoding and or decoding systems can be embodied as one or more application specific integrated circuits (ASICs) or microcontrollers that can be programmed to carry out one or more of the systems and procedures described herein. Certain terms are used throughout the description and claims refer to particular system components. As one skilled in the art will appreciate, components may be referred to by different names. This document does not intend to distinguish between components that differ in name, but not function.

One skilled in the art will recognize that the Internet service may be configured to provide Internet access to one or more computing devices that are coupled to the Internet service, and that the computing devices may include one or more processors, buses, memory devices, display devices, input/output devices, and the like. Furthermore, those skilled in the art may appreciate that the Internet service may be coupled to one or more databases, repositories, servers, and the like, which may be utilized in order to implement any of the embodiments of the disclosure as described herein.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

It is noted at the outset that the terms “coupled,” “connected”, “connecting,” “mechanically connected,” etc., are used interchangeably herein to generally refer to the condition of being mechanically/physically connected. The terms “couple” and “coupling” are also used in a non-mechanical/physical context that refers to absorption of microwave energy by a material. It is further noted that various figures (including component diagrams) shown and discussed herein are for illustrative purpose only, and are not drawn to scale.

If any disclosures are incorporated herein by reference and such incorporated disclosures conflict in part and/or in whole with the present disclosure, then to the extent of conflict, and/or broader disclosure, and/or broader definition of terms, the present disclosure controls. If such incorporated disclosures conflict in part and/or in whole with one another, then to the extent of conflict, the later-dated disclosure controls.

The terminology used herein can imply direct or indirect, full or partial, temporary or permanent, immediate or delayed, synchronous or asynchronous, action or inaction. For example, when an element is referred to as being “on,” “connected” or “coupled” to another element, then the element can be directly on, connected or coupled to the other element and/or intervening elements may be present, including indirect and/or direct variants. In contrast, when an element is referred to as being “directly connected” or “directly coupled” to another element, there are no intervening elements present.

Although the terms first, second, etc. may be used herein to describe various elements, components, regions, layers and/or sections, these elements, components, regions, layers and/or sections should not necessarily be limited by such terms. These terms are only used to distinguish one element, component, region, layer or section from another element, component, region, layer or section. Thus, a first element, component, region, layer or section discussed below could be termed a second element, component, region, layer or section without departing from the teachings of the present disclosure.

The terminology used herein is for the purpose of describing particular embodiments only and is not intended to be necessarily limiting of the disclosure. As used herein, the singular forms “a,” “an”, and “the” are intended to include the plural forms as well, unless the context clearly indicates otherwise. The terms “comprises,” “includes” and/or “comprising,” “including” when used in this specification, specify the presence of stated features, integers, steps, operations, elements, and/or components, but do not preclude the presence or addition of one or more other features, integers, steps, operations, elements, components, and/or groups thereof.

Furthermore, relative terms such as “below,” “lower,” “above,” and “upper” may be used herein to describe one element's relationship to another element as illustrated in the accompanying drawings. Such relative terms are intended to encompass different orientations of illustrated technologies in addition to the orientation depicted in the accompanying drawings. For example, if a device in the accompanying drawings is turned over, then the elements described as being on the “lower” side of other elements would then be oriented on “upper” sides of the other elements. Similarly, if the device in one of the figures is turned over, elements described as “below” or “beneath” other elements would then be oriented “above” the other elements. Therefore, the example terms “below” and “lower” can, therefore, encompass both an orientation of above and below.

Example embodiments of the present disclosure are described herein with reference to illustrations of idealized embodiments (and intermediate structures) of the present disclosure. As such, variations from the shapes of the illustrations as a result, for example, of manufacturing techniques and/or tolerances, are to be expected. Thus, the example embodiments of the present disclosure should not be construed as necessarily limited to the particular shapes of regions illustrated herein, but are to include deviations in shapes that result, for example, from manufacturing.

Any and/or all elements, as disclosed herein, can be formed from a same, structurally continuous piece, such as being unitary, and/or be separately manufactured and/or connected, such as being an assembly and/or modules. Any and/or all elements, as disclosed herein, can be manufactured via any manufacturing processes, whether additive manufacturing, subtractive manufacturing and/or other any other types of manufacturing. For example, some manufacturing processes include three-dimensional (3D) printing, laser cutting, computer numerical control (CNC) routing, milling, pressing, stamping, vacuum forming, hydroforming, injection molding, lithography and/or others.

Any and/or all elements, as disclosed herein, can include, whether partially and/or fully, a solid, including a metal, a mineral, a ceramic, an amorphous solid, such as glass, a glass ceramic, an organic solid, such as wood and/or a polymer, such as rubber, a composite material, a semiconductor, a nano-material, a biomaterial and/or any combinations thereof. Any and/or all elements, as disclosed herein, can include, whether partially and/or fully, a coating, including an informational coating, such as ink, an adhesive coating, a melt-adhesive coating, such as vacuum seal and/or heat seal, a release coating, such as tape liner, a low surface energy coating, an optical coating, such as for tint, color, hue, saturation, tone, shade, transparency, translucency, non-transparency, luminescence, anti-reflection and/or holographic, a photo-sensitive coating, an electronic and/or thermal property coating, such as for passivity, insulation, resistance or conduction, a magnetic coating, a water-resistant and/or waterproof coating, a scent coating and/or any combinations thereof.

Unless otherwise defined, all terms (including technical and scientific terms) used herein have the same meaning as commonly understood by one of ordinary skill in the art to which this disclosure belongs. The terms, such as those defined in commonly used dictionaries, should be interpreted as having a meaning that is consistent with their meaning in the context of the relevant art and should not be interpreted in an idealized and/or overly formal sense unless expressly so defined herein.

While various embodiments have been described above, it should be understood that they have been presented by way of example only, and not limitation. The descriptions are not intended to limit the scope of the invention to the particular forms set forth herein. To the contrary, the present descriptions are intended to cover such alternatives, modifications, and equivalents as may be included within the spirit and scope of the invention as defined by the appended claims and otherwise appreciated by one of ordinary skill in the art. Thus, the breadth and scope of a preferred embodiment should not be limited by any of the above-described exemplary embodiments.

What is claimed is:

1. A digital anthropometer, comprising:
a rotary encoder;
an enclosure housing the rotary encoder;
a wheel associated with the enclosure and rotatably coupled to the rotary encoder for measuring a displacement as the wheel rolls along a surface;
a digital display configured to show the displacement as a measurement value; and
at least one control button positioned on the enclosure and configured to operate the rotary encoder.

2. The digital anthropometer of claim 1, further comprising a rail engagement interface, the rail engagement interface including:
rollers rotatably supported on the enclosure, wherein the rollers are configured to engage with a measurement rail and support the digital anthropometer as it travels along the measurement rail; and
wherein the wheel is further configured to roll along the measurement rail, the measurement rail being placed in proximity to a body or body part of a user.

3. The digital anthropometer of claim 1, further comprising a short-range wireless communications module, wherein the short-range wireless communications module is configured to wirelessly transmit the measurement value to an external device, wherein the short-range wireless communications module comprises Bluetooth and is configured to transmit data to the external device in real time.

4. The digital anthropometer of claim 1, further comprising a second digital display positioned on an opposite side of the enclosure, the second digital display configured to provide visibility of the measurement value from multiple angles.

5. The digital anthropometer of claim 1, wherein the at least one control button includes:
a unit toggle button configured to switch between different units of the measurement value;
a hold button configured to freeze the measurement value displayed on the digital display; and
a zero button configured to reset the measurement value to zero.

6. The digital anthropometer of claim 1, further comprising a pressure sensor associated with the rotary encoder, the pressure sensor configured to sense an amount of force applied to the wheel during measurement.

7. The digital anthropometer of claim 1, wherein the rotary encoder is configured to measure displacement with a resolution of 0.1 millimeters.

8. The digital anthropometer of claim 1, further comprising a detachable component configured to enable switching between anthropometry and skinfold measurement modes.

9. The digital anthropometer of claim 1, further comprising a sensor configured to indicate a horizontal plane orientation of the digital anthropometer.

10. A method of using a digital anthropometer, the method comprising:
positioning a wheel on a surface to be measured;
rolling the wheel along the surface to measure a displacement;
converting the displacement into a distance using a rotary encoder coupled to the wheel;
displaying the distance on a digital display, the displayed distance being indicative of the displacement; and
transmitting the distance wirelessly to an external device via a Bluetooth module.

11. The method of claim 10, further comprising switching between different units of measurement by toggling a unit button.

12. The method of claim 10, further comprising freezing the displayed measurement value by pressing a hold button.

13. The method of claim 10, further comprising resetting the displayed measurement value to zero by pressing a zero button.

14. The method of claim 10, further comprising sensing an amount of force applied during measurement using a pressure sensor.

15. The method of claim 10, further comprising providing visibility of the measurement value from multiple angles using a second digital display.

16. The method of claim 10, further comprising switching between anthropometry and skinfold measurement modes by attaching or detaching a detachable component.

17. The method of claim 10, further comprising indicating a horizontal plane orientation of the digital anthropometer using an output from a sensor.

18. A digital anthropometer, comprising:
an enclosure housing a rotary encoder;
a wheel associated with the enclosure and rotatably coupled to the rotary encoder, the wheel configured to measure a displacement as the wheel rolls along a body part of a user to measure a length of the body part;
a pressure sensor associated with the rotary encoder;
a microcontroller configured to:
receive output from the rotary encoder indicative of the wheel rolling against the body part;
receive a pressure signal from the pressure sensor while the wheel is used against the body part; and
determine a measurement value corresponding to the length of the body part based on rotations of the wheel as determined from the output of the rotary encoder.

19. The digital anthropometer of claim 18, further comprising a support arm extending from the enclosure.

20. The digital anthropometer of claim 18, wherein the pressure sensor is configured to provide real-time feedback to the microcontroller, allowing for dynamic adjustment of the displayed measurement value based on detected pressure to enhance measurement accuracy, and wherein the microcontroller is further configured to generate an alert via the digital display or an auditory signal when an applied pressure exceeds a predefined threshold, thereby indicating that excessive force is being applied to avoid distorting the measurement value.
